(19) United States
(12) Patent Application Publication
(10) Pub. No.: US 2021/0219579 A1
(43) Pub. Date: Jul. 22, 2021

(54) COMPOSITIONS AND METHODS FOR NOURISHING MAMMALS

(71) Applicant: KALMARNA LIMITED, Tortola (VG)

(72) Inventors: Alon ROSENBERG, Rehovot (IL); Abraham MILSTEIN, Rehovot (IL); Ava Marie FIRTH, West Bridgford, Nottinghamshire (GB); Simon VAN DALSEM, Alkmaar (NL); Arie HALPERN, Pully (CH)

(21) Appl. No.: 17/051,594

(22) PCT Filed: Apr. 29, 2019
(86) PCT No.: PCT/IL2019/050472

§ 371 Date: Oct. 29, 2020

Related U.S. Application Data
(60) Provisional application No. 62/664,174, filed on Apr. 29, 2018.
(60) Provisional application No. 62/723,496, filed on Aug. 28, 2018.

(51) Int. Cl. A23L 2/60; A23L 2/68; A23L 33/125; A23L 33/16; A23L 33/175; A23K 20/142; A23K 20/22; A23K 20/26; A23K 20/163

(52) U.S. Cl. CPC A23L 2/60; A23L 2/68; A23L 33/125; A23L 33/16; A23K 20/163; A23K 20/142; A23K 20/22; A23K 20/26; A23L 33/175

(57) ABSTRACT

The present invention discloses palatable oral formulations for hydrating, supporting gut activity, micronutrient supplementation and providing oral carriers for vaccines, antibiotics and food supplements, of inter alia, companion or pet animals.

### FIELD OF THE INVENTION

The invention generally pertains to compositions and method for nourishing mammals.

### BACKGROUND OF THE INVENTION

Physical activity in hot environments can increase the risk of heat stress or heat stroke in pets. Pets, or companion animals, are affected by high temperatures and humidity; thus heat tolerance is influenced by acclimatization to the environment, physical fitness, and hydration state.

Since dogs have minimal sweating capacity, thermoregulation relies primarily on evaporative mechanisms through panting. Given dogs' inability to sweat (except through the pads of their feet), dogs rely on panting to cool them down and high temperatures, coupled with a lack of water, can severely interfere with this process, leading to dehydration. High humidity can also negatively affect a canine's capacity for evaporative cooling. Even in moderate temperatures, dogs can even suffer from dehydration; see Sodhi, Nidhi. “Dog hydration strategies.” Aust Vet J 2018; 96(3):N20 (2018), incorporated herein as a reference.

In addition to the environmental factors of heat and humidity, the dog's adaptation to environmental conditions (acclimatization), overall fitness/conditioning and state of hydration are thought to be major factors that impact heat tolerance. Canine thermoregulation involves the inhalation of cooler, dryer air through the nose and mouth, which causes evaporative heat loss from the nasal and oral mucosa and tongue, and the exhalation of hotter, moister air. In conditions in which the rate of heat generation is greater than the rate of heat dissipation, increased salivation and lingual blood flow allow for convection and evaporative cooling). Evaporative cooling results in electrolyte-free water loss (estimated at ˜10 mL/kg/h) which can contribute to dehydration, see Otto, Cynthia M., et al. “evaluation of three hydration strategies in Detection Dogs Working in a hot environment.” Frontiers in veterinary science 4 (2017), incorporated herein as a reference.

Dehydration in horses is a major concern for those involved in the equine industry, especially during the hot summer months. At these times high temperatures can induce increased sweating rates in horses, potentially causing water and electrolyte losses to reach dangerous levels, see Green, B. L., and J. L. Wahrmund. “143 Impact of Hydration Supplements on Blood Electrolyte Concentrations of Exercised Horses. During the Summer” Journal of Animal Science94.suppl_1 (2016): 69-70., incorporated herein as a reference.

The domestic cat is adapted to retain water by producing urine which is very concentrated compared to most other mammals. Producing highly concentrated urine, however, can have deleterious effects, such as enhancing development of urinary stones and other less well defined urinary tract conditions such feline idiopathic cystitis. Manipulating hydration is the most appealing approach to prevent dehydration and increase heat tolerance and workability. Prior documents disclose methods and hydration strategies for pets: Palatability enhancers for pet food are known in the art. U.S. Pat. Nos. 6,354,920 and 6,350,485 to Brunner, incorporated herein as a reference, disclose the use of tetrasodium pyrophosphate as a palatability enhancer for dry and semi-moist cat food. U.S. Pat. No. 5,186,964 to Gierhart et al. discloses the use of a pyrophosphate salt or an acid phosphate salt as a palatability enhancer for dry pet food. U.S. Pat. No. 7,244,460 to Lee et al. discloses the use of a tripolyphosphate salt as a palatability enhancer for dry and semi-dry pet food. The use of a palatability enhancer as a flavoured drinking water additive is discloses in US patent application 20140127352 to Woodward et al. US patent application 20140127352 to Woodward et al discloses flavoured drinking water additives and flavoured drinking water compositions, as well as methods for increasing water consumption by an animal and improving hydration for animals using the flavoured drinking water additives and flavoured drinking water compositions of Woodward invention based on pyrophosphate salts. US patent application 20180085309 to Baskins incorporated herein as a reference discloses a pet hydration System, comprising of specific ranges of vitamins and minerals conducive to a pet's health, with such nutrients placed into a liquid beverage and ultimately dispensed via a compartmentalized beverage dispenser. The compartmentalized beverage dispenser is formed such that separate liquids are measured out and dispensed to the pet in a manner that the pet receives the optimal amount of nutrition. US patent application US20170172180 to Znaghi discloses composition and methods for improving hydration and water intake in an animal. In one embodiment, a hydration composition can comprise water and a hydration additive, where the hydration additive consists essentially of a sugar alcohol and a protein, where the sugar alcohol includes glycerol and the protein includes whey. Additionally, a method of improving hydration and water intake in an animal can comprise administering the hydration composition with water to the animal. U.S. Pat. No. 9,884,035B2 to Brockman et al., discloses methods and compositions for improving the level of hydration in a cat. Cats fed diets containing certain amounts and ratios of arachidonic acid and eicosapentaenoic acid will be sufficiently hydrated and at reduced risk for development of urinary stones, feline idiopathic cystitis.

Mink have been farmed for fur in the United States for 130 years. In 2010, the U.S. ranked fifth in production behind Denmark, China, the Netherlands, and Poland. Mink typically breed in March and give birth to their litters in May. (see Wikipedia, a “fur faming”, incorporated herein as a reference).

Mortality in mink kits held under conventional farm conditions is believed to be related to dehydration, see Brink, A-L., Leif Lau Jeppesen, and Knud Erik Heller. “Behaviour in suckling mink kits under farm conditions: effects of accessibility of drinking water.” Applied Animal Behaviour Science 89.1 (2004): 131-137; incorporated herein as a reference.

As shown above, physical activity is known to increase an individual's water requirement, particularly while exercising. Additionally, stress related to travel, boarding, or illness also compromises hydration status in cats, minks and dogs and horses. Thus, there remains a need for compositions and methods to improve water intake and increase hydration in companion animals such as dogs, cats and horses.

### BRIEF DESCRIPTION OF THE FIGURES

The accompanying drawings, which are included to provide a further understanding of the invention and are incorporated in and constitute a part of this specification, illustrate embodiments of the invention and together with the description serve to explain the principles of the invention

FIG. 1 depicting a non-limiting example of a preferred gel embodiment of the current invention; and

FIG. 2 depicting yet another non-limiting example of a preferred gel embodiment of the current invention.

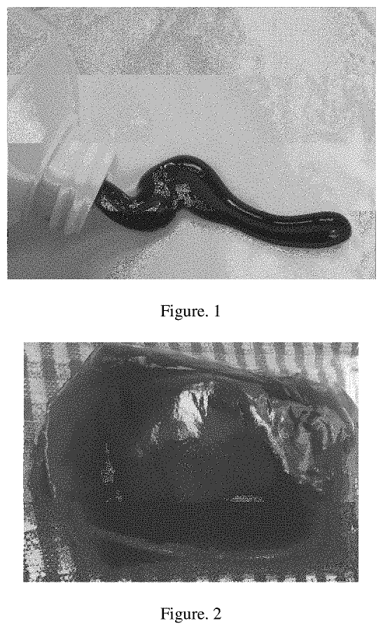

### SUMMARY OF THE INVENTION

It is one object of the invention to disclose an oral isotonic compositions characterized by no residual chlorine taste for the hydration of mammals comprising at least three members of a group consisting of steviol glycoside, citric acid monohydrate, monosodium glutamate, glycine, mixtures and derivatives thereof.

Chlorinous flavours at the food and drinks are the leading cause of human and animals' complaints, dissatisfaction and dehydration, see e.g., Piriou, Philippe, et al. “Chlorinous flavour perception in drinking water.” Water Science and Technology 49.9 (2004): 321-328. A chlorine-free taste in foods and drinks are hence a long felt need.

It is another object of the invention to disclose an oral isotonic compositions characterized by no residual chlorine taste for the hydration of mammals comprising (a) at least three members of a group consisting of steviol glycoside, citric acid monohydrate, monosodium glutamate, glycine, mixtures and derivatives thereof; and (b) prebiotic any derivatives and mixtures thereof.

Prebiotics are food ingredients that induce the growth or activity of beneficial microorganisms (e.g., bacteria and fungi). The term ‘prebiotic’ hence refers hereinafter, inter alia and in a non-limiting manner, to both of endogenous and exogenous sources-driven prebiotics, and to at least one member of a group consisting of edibles being non-digestible and resistant to breakdown by stomach acid and enzymes in the human gastrointestinal tract; edibles selectively fermented by intestinal microorganisms; and edibles selectively target and stimulate the growth and activity of beneficial bacteria; see Hutkins R W et al., (2016). “Prebiotics: why definitions matter”. Curr Opin Biotechnol. 37: 1-7. The term also refers to synbiotics, e.g., food ingredients or dietary supplements combining probiotics and prebiotics in a form of synergism, see DeVrese M, Schrezenmeir J (2008) Probiotics, prebiotics, and synbiotics in food biotechnology (pp. 1-66). Springer Berlin Heidelberg which is incorporated herein as a reference.

It is another object of the invention to disclose an isotonic drink characterized by no residual chlorine taste for the hydration of mammals comprising at least three members of a group consisting of steviol glycoside, citric acid monohydrate, monosodium glutamate, glycine, mixtures and derivatives thereof; and any derivatives and mixtures thereof.

It is another object of the invention to disclose an isotonic drink compositions characterized by no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose an apple flavoured horses' isotonic drink characterized by no residual chlorine taste comprising (a) at least three members of a group consisting steviol glycoside, citric acid monohydrate, monosodium glutamate, glycine, mixtures and derivatives thereof; and (b) an apple-flavouring agent.

The term ‘apple-flavouring agent’ refers herein after, inter alia and in a non-limiting manner, to one or more of the following: apple extracts, apple flavour No. 64625, malic acids, esters, salts and derivatives thereof, apple flavour fragrance, e.g., as defined in CN patent applications 107279615, 104830607 and 105212170, JP patent application 2011152095, Maxarome AC 1632, all are incorporated herein as references, taste enhancers, emulsifiers and stabilizers thereof.

Maxarome AC 1632 is a concentrated, high performance flavour developed specifically for inclusion into animal feeds where it will improve palatability and encourage feed intake. Maxarome AC 1632 includes Vanillin and other aldehydes, together with mixed esters. Maxarome AC 1632 has the following characteristics: Physical Appearance: Beige-pale brown free flowing powder; Volatile Matter: 38.94%; Solubility: Insoluble; Bulk Density: 0.625 g/cm3; Flavour Classification: Nature Identical; Flavour Type: Spicy Apple; Product Classification: Conforms to EU Regulation 1831/2003—Annex 1, 2(b); Pre-mixture of flavouring compounds; Added Colouring: None Added; Antioxidants: None Added.

It is another object of the invention to disclose an apple flavoured horses' isotonic drink characterized by no residual chlorine taste comprising (a) at least three members of a group consisting steviol glycoside, citric acid monohydrate, monosodium glutamate, glycine, mixtures and derivatives thereof; (b) An apple-flavouring agent; and (c) prebiotic.

It is another object of the invention to disclose an isotonic taste enhancer, e.g., fluid, solid or gel-formed taste enhancer, edible meal topper, a treat tec., characterized by no residual chlorine taste for the hydration of mammals comprising at least three members of a group consisting steviol glycoside, citric acid monohydrate, monosodium glutamate, glycine, mixtures and derivatives thereof.

It is another object of the invention to disclose an isotonic taste enhancer characterized by no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose an isotonic taste enhancer characterized by no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an isotonic taste enhancer compositions characterized by no residual chlorine taste for the hydration of mammals comprising (a) at least three members of a group consisting steviol glycoside, citric acid monohydrate, monosodium glutamate, glycine, mixtures and derivatives thereof; and (b) prebiotic.

It is another object of the invention to disclose an isotonic apple flavoured horses' taste enhancer characterized by no residual chlorine taste comprising (a) at least three members of a group consisting steviol glycoside, citric acid monohydrate, monosodium glutamate, glycine, mixtures and derivatives thereof; and (b) an apple-flavouring agent.

It is another object of the invention to disclose an isotonic Apple flavoured horses' taste enhancer characterized by no residual chlorine taste comprising (a) at least three members of a group consisting steviol glycoside, citric acid monohydrate, monosodium glutamate, glycine, mixtures and derivatives thereof; (b) an apple-flavouring agent; and (c) prebiotic.

It is another object of the invention to disclose an edible powder characterized by no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an edible powder characterized by no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose an edible powder characterized by no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an edible powder compositions characterized by no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose an apple flavoured horses' edible powder characterized by no residual chlorine taste comprising (a) at least three members of a group consisting steviol glycoside, citric acid monohydrate, monosodium glutamate, glycine, mixtures and derivatives thereof; and (b) an apple-flavouring agent.

It is another object of the invention to disclose an apple flavoured horses' edible powder characterized by no residual chlorine taste comprising (a) at least three members of a group consisting steviol glycoside, citric acid monohydrate, monosodium glutamate, glycine, mixtures and derivatives thereof; (b) An apple-flavouring agent; and (c) prebiotic.

It is another object of the invention to disclose an edible gel characterized by no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an edible gel compositions characterized by no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose an edible isotonic gel characterized by no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an edible isotonic gel compositions characterized by no residual chlorine taste for the hydration of mammals comprising (a) at least three members of a group consisting steviol glycoside, citric acid monohydrate, monosodium glutamate, glycine, mixtures and derivatives thereof and (b) prebiotic any derivatives and mixtures thereof.

It is another object of the invention to disclose an apple flavoured horses' edible isotonic gel characterized by no residual chlorine taste comprising (a) at least three members of a group consisting steviol glycoside, citric acid monohydrate, monosodium glutamate, glycine, mixtures and derivatives thereof; and (b) an apple-flavouring agent.

It is another object of the invention to disclose an apple flavoured horses' edible isotonic gel characterized by no residual chlorine taste comprising (a) at least three members of a group consisting steviol glycoside, citric acid monohydrate, monosodium glutamate, glycine, mixtures and derivatives thereof; (b) an apple-flavouring agent; and (c) prebiotic.

It is another object of the invention to disclose a solid shear thinning thixotropic edible gel characterized by no residual chlorine taste for the hydration of mammals comprising at least three members of a group consisting of steviol glycoside, citric acid monohydrate, monosodium glutamate, glycine, mixtures and derivatives thereof.

It is another object of the invention to disclose a solid shear thinning thixotropic edible gel characterized by no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose an apple flavoured horses' solid shear thinning thixotropic edible gel characterized by no residual chlorine taste comprising (a) at least three members of a group consisting steviol glycoside, citric acid monohydrate, monosodium glutamate, glycine, mixtures and derivatives thereof; and (b) an apple-flavouring agent.

It is another object of the invention to disclose an apple flavoured horses' solid shear thinning thixotropic edible gel characterized by no residual chlorine taste comprising (a) at least three members of a group consisting steviol glycoside, citric acid monohydrate, monosodium glutamate, glycine, mixtures and derivatives thereof; (b) an apple-flavouring agent; and (c) prebiotic.

It is another object of the invention to disclose an oral isotonic compositions characterized by no residual chlorine taste for the hydration of mammals comprising (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

It is another object of the invention to disclose an oral isotonic compositions characterized by no residual chlorine taste for the hydration of mammals comprising (a) a member of a group consisting of vaccines, iron-containing compositions, minerals, salts, amino acids, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) prebiotic any derivatives and mixtures thereof.

It is another object of the invention to disclose an isotonic drink characterized by no residual chlorine taste for the hydration of mammals comprising (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof; and any derivatives and mixtures thereof.

It is another object of the invention to disclose an isotonic drink compositions characterized by no residual chlorine taste for the hydration of mammals comprising (a) a member of a group consisting of vaccines, iron-containing compositions, minerals, salts, amino acids, medicaments, vitamins, nutraceuticals, antibiotics and food supplements; and (b) at least three members of a group consisting steviol glycoside, citric acid monohydrate, monosodium glutamate, glycine, mixtures and derivatives thereof; and (c) prebiotic any derivatives and mixtures thereof.

It is another object of the invention to disclose an apple flavoured horses' isotonic drink characterized by no residual chlorine taste comprising (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) an apple-flavouring agent.

It is another object of the invention to disclose an apple flavoured horses' isotonic drink characterized by no residual chlorine taste comprising (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.

It is another object of the invention to disclose an edible powder characterized by no residual chlorine taste for the hydration of mammals comprising (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

It is another object of the invention to disclose an edible powder characterized by no residual chlorine taste for the hydration of mammals comprising (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic any derivatives and mixtures thereof.

It is another object of the invention to disclose an edible powder characterized by no residual chlorine taste for the hydration of mammals comprising (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof.

It is another object of the invention to disclose an edible powder compositions characterized by no residual chlorine taste for the hydration of mammals comprising (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic.

It is another object of the invention to disclose an apple flavoured horses' edible powder characterized by no residual chlorine taste comprising (a) a member of a group consisting of vaccines, iron-containing compositions, minerals, salts, amino, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) An apple-flavouring agent.

It is another object of the invention to disclose an apple flavoured horses' edible powder characterized by no residual chlorine taste comprising (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.

It is another object of the invention to disclose an oral isotonic compositions characterized by chicken flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an oral isotonic compositions characterized by chicken flavour and no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose isotonic drink characterized by chicken flavour and no residual chlorine taste for the hydration of mammals comprising at least three members of a group consisting of steviol glycoside, citric acid monohydrate, monosodium glutamate, glycine, mixtures and derivatives thereof; and any derivatives and mixtures thereof.

It is another object of the invention to disclose an isotonic drink compositions characterized by chicken flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an isotonic taste enhancer characterized by chicken flavour and no residual chlorine taste for the hydration of mammals comprising at least three members of a group consisting steviol glycoside, citric acid monohydrate, monosodium glutamate, glycine, mixtures and derivatives thereof.

It is another object of the invention to disclose an isotonic taste enhancer characterized by chicken flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an edible powder characterized by chicken flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an edible powder characterized by chicken flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an edible gel characterized by chicken flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an edible gel compositions characterized by chicken flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an edible isotonic gel characterized by chicken flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an edible isotonic gel compositions characterized by chicken flavour and no residual chlorine taste for the hydration of mammals comprising (a) at least three members of a group consisting steviol glycoside, citric acid monohydrate, monosodium glutamate, glycine, mixtures and derivatives thereof and (b) prebiotic any derivatives and mixtures thereof.

It is another object of the invention to disclose a solid shear thinning thixotropic edible gel characterized by chicken flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose a solid shear thinning thixotropic edible gel characterized by chicken flavour and no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose an oral isotonic compositions characterized by fish flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an oral isotonic compositions characterized by fish flavour and no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose an isotonic drink characterized by fish flavour and no residual chlorine taste for the hydration of mammals comprising at least three members of a group consisting of steviol glycoside, citric acid monohydrate, monosodium glutamate, glycine, mixtures and derivatives thereof; and any derivatives and mixtures thereof.

It is another object of the invention to disclose an isotonic drink compositions characterized by fish flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an isotonic taste enhancer characterized by fish flavour and no residual chlorine taste for the hydration of mammals comprising at least three members of a group consisting steviol glycoside, citric acid monohydrate, monosodium glutamate, glycine, mixtures and derivatives thereof.

It is another object of the invention to disclose an isotonic taste enhancer characterized by fish flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an edible powder characterized by fish flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an edible powder characterized by fish flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an edible gel characterized by fish flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an Edible gel compositions characterized by fish flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an edible isotonic gel characterized by fish flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an edible isotonic gel compositions characterized by fish flavour and no residual chlorine taste for the hydration of mammals comprising (a) at least three members of a group consisting steviol glycoside, citric acid monohydrate, monosodium glutamate, glycine, mixtures and derivatives thereof and (b) prebiotic any derivatives and mixtures thereof.

It is another object of the invention to disclose an solid shear thinning thixotropic edible gel characterized by fish flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an solid shear thinning thixotropic edible gel characterized by fish flavour and no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose an oral isotonic compositions characterized by beef flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an oral isotonic compositions characterized by beef flavour and no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.

It is another object of the invention to disclose an isotonic drink characterized by beef flavour and no residual chlorine taste for the hydration of mammals comprising at least three members of a group consisting of steviol glycoside, citric acid monohydrate, monosodium glutamate, glycine, mixtures and derivatives thereof; and any derivatives and mixtures thereof.

It is another object of the invention to disclose an isotonic drink compositions characterized by beef flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an isotonic taste enhancer characterized by beef flavour and no residual chlorine taste for the hydration of mammals comprising at least three members of a group consisting steviol glycoside, citric acid monohydrate, monosodium glutamate, glycine, mixtures and derivatives thereof.

It is another object of the invention to disclose an isotonic taste enhancer characterized by beef flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an edible powder characterized by beef flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an edible powder characterized by beef flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an edible gel characterized by beef flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an edible gel compositions characterized by beef flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.

It is another object of the invention to disclose an edible isotonic gel characterized by beef flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose an edible isotonic gel compositions characterized by beef flavour and no residual chlorine taste for the hydration of mammals comprising (a) at least three members of a group consisting steviol glycoside, citric acid monohydrate, monosodium glutamate, glycine, mixtures and derivatives thereof and (b) prebiotic any derivatives and mixtures thereof.

It is another object of the invention to disclose a solid shear thinning thixotropic edible gel characterized by beef flavour and no residual chlorine taste for the hydration of mammals comprising at least three members of a group consisting of steviol glycoside, citric acid monohydrate, monosodium glutamate, glycine, mixtures and derivatives thereof.

It is another object of the invention to disclose a solid shear thinning thixotropic edible gel characterized by beef flavour and no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.

It is still another object of the invention to disclose a method selected from a group consisting of enhancing palatability, fast hydrating, gut activity supporting, providing micro-nutrition supplementation, and enhancing digestion and absorption; t; wherein said method comprising step of administrating a mammal a composition as defined in any of the above.

### DETAILED DESCRIPTION OF THE PREFERRED EMBODIMENTS

The following description is provided, alongside all chapters of the present invention, so as to enable any person skilled in the art to make use of said invention and sets forth the best modes contemplated by the inventor of carrying out this invention. Various modifications, however, are adapted to remain apparent to those skilled in the art, since the generic principles of the present invention have been defined specifically to provide compositions and methods method for orally nourishing mammalians. Specifically, the present invention discloses palatable oral formulations for hydrating, supporting gut activity, micronutrient supplementation and providing oral carriers for vaccines, antibiotics and food supplements, of inter alia, companion or pet animals.

As used herein after, the terms “pet” or “companion animal” interchangeably refers hereinafter to an animal kept primarily for a person's company, protection, or entertainment rather than as a working animal, livestock, or laboratory animal. Popular pets are often noted for their attractive appearances, intelligence, and relatable personalities. Two of the most popular pets are dogs and cats. Other animals commonly kept include: pigs, ferrets, rabbits; rodents, such as gerbils, hamsters, chinchillas, rats, and guinea pigs; avian pets, such as parrots, passerines, and fowl; reptile pets, such as turtles, lizards and snakes; aquatic pets, such as fish, freshwater and saltwater snails, and frogs; and arthropod pets, such as tarantulas and hermit crabs.

As used hereinafter, the term “steviol” generally refers to steviol glycoside, Rebaudioside A extract.

The current invention discloses oral chlorine-free compositions for hydration of humans and animals, characterized by enhanced palatability, fast hydration, gut support activity, micro-nutrition supplementation, and enlaced digestion and absorption. It is in the scope of the invention wherein aforesaid non-genetically-modified compositions, products and formulations comprises or based on commercially available Px (trade mark) by Tonisity International Ltd, namely, a composition, e.g., defined below:

TABLE 1
Ingredients of Px formulation.

| Ingredients | (%) |
|---|---|
| glucose anhydrous | 1.5540 |
| monosodium glutamate | 0.3900 |
| sodium chloride, vacuum dried | 0.2201 |
| citric acid anhydrous | 0.1860 |
| potassium chloride | 0.1500 |
| glycine | 0.1500 |
| hydrolyzed whey protein isolate | 0.1500 |
| monosodium dihydrogen phosphate | 0.1000 |
| xanthan gum | 0.0500 |
| 1-glutamic acid | 0.0400 |
| stevia (min. 85% steviol glycoside,, Rebaudioside A extract.) | 0.0100 |
| ingredients | 3.0000 |
| demineralized water | 97.000 |

The composition consists at least two members of a group consisting of steviol glycoside, citric acid monohydrate, monosodium glutamate and glycine, this composition characterized by that that the composition consists at least three members of a group consisting of steviol glycoside, citric acid monohydrate, monosodium glutamate and glycine. Additionally or alternatively, this composition is characterized by that that said composition consisting of steviol glycoside, citric acid monohydrate, monosodium glutamate and glycine.

Additionally or alternatively the composition of the current invention also discloses a flavouring agent selected form a group consisting of apple, chicken, beef or fish flavours, with or without prebiotics, and any derivatives and mixtures thereof.

Additionally or alternatively the composition of the current invention is a palatable carrier of at least one compound of a group consisting of vaccines, iron-containing compositions, minerals, salts, amino acids, medicaments, vitamins, nutraceuticals, antibiotics and food supplements, for nourishing and treating mammals. Thus, the composition of the current invention overcomes the risks of needle administration of the compounds, decreases labor and manpower needed for injecting the treated mammals, and increasing compliance to treatment.

The current invention discloses compositions comprising isotonic drinks (ready-to-use liquids), isotonic edible powders, isotonic taste enhancer, e.g., fluid, edible solid or gel-formed taste enhancer, edible meal toppers (“Gravy”), a treat etc., characterized by no residual chlorine taste for the hydration of mammals, such as cats, dogs, horses, and mink.

These compositions of the current invention compositions comprise:

Equine formulations: formulations for feeding horses of all type of services: horses for work, sport entrainment, culture, warfare, and for therapeutic uses.

These equine formulations comprise e.g., one of the followings:
a. An apple flavoured horses' isotonic drink characterized by no residual chlorine taste comprising (a) at least three members of a group consisting steviol glycoside, citric acid monohydrate, monosodium glutamate, glycine, mixtures and derivatives thereof; and (b) an apple-flavouring agent.
b. An apple flavoured horses' isotonic drink characterized by no residual chlorine taste comprising (a) at least three members of a group consisting steviol glycoside, citric acid monohydrate, monosodium glutamate, glycine, mixtures and derivatives thereof; (b) An apple-flavouring agent; and (c) prebiotic.
c. An isotonic apple flavoured horses' taste enhancer characterized by no residual chlorine taste comprising (a) at least three members of a group consisting steviol glycoside, citric acid monohydrate, monosodium glutamate, glycine, mixtures and derivatives thereof; and (b) an apple-flavouring agent.
d. An isotonic Apple flavoured horses' taste enhancer characterized by no residual chlorine taste comprising (a) at least three members of a group consisting steviol glycoside, citric acid monohydrate, monosodium glutamate, glycine, mixtures and derivatives thereof; (b) an apple-flavouring agent; and (c) prebiotic.
e. An apple flavoured horses' edible powder characterized by no residual chlorine taste comprising (a) at least three members of a group consisting steviol glycoside, citric acid monohydrate, monosodium glutamate, glycine, mixtures and derivatives thereof; and (b) an apple-flavouring agent.
f. An apple flavoured horses' edible powder characterized by no residual chlorine taste comprising (a) at least three members of a group consisting steviol glycoside, citric acid monohydrate, monosodium glutamate, glycine, mixtures and derivatives thereof; (b) An apple-flavouring agent; and (c) prebiotic.
g. An apple flavoured horses' edible isotonic gel characterized by no residual chlorine taste comprising (a) at least three members of a group consisting steviol glycoside, citric acid monohydrate, monosodium glutamate, glycine, mixtures and derivatives thereof; and (b) an apple-flavouring agent.
h. An apple flavoured horses' edible isotonic gel characterized by no residual chlorine taste comprising (a) at least three members of a group consisting steviol glycoside, citric acid monohydrate, monosodium glutamate, glycine, mixtures and derivatives thereof; (b) an apple-flavouring agent; and (c) prebiotic.
i. An apple flavoured horses' solid shear thinning thixotropic edible gel characterized by no residual chlorine taste comprising (a) at least three members of a group consisting steviol glycoside, citric acid monohydrate, monosodium glutamate, glycine, mixtures and derivatives thereof; and (b) an apple-flavouring agent.
j. An apple flavoured horses' solid shear thinning thixotropic edible gel characterized by no residual chlorine taste comprising (a) at least three members of a group consisting steviol glycoside, citric acid monohydrate, monosodium glutamate, glycine, mixtures and derivatives thereof; (b) an apple-flavouring agent; and (c) prebiotic.
k. An apple flavoured horses' isotonic drink characterized by no residual chlorine taste comprising (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) an apple-flavouring agent.
l. An apple flavoured horses' isotonic drink characterized by no residual chlorine taste comprising (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) An apple-flavouring agent; and (d) prebiotic.

m. An apple flavoured horses' edible powder characterized by no residual chlorine taste comprising (a) a member of a group consisting of vaccines, iron-containing compositions, minerals, salts, amino, medicaments, vitamins, nutraceuticals, antibiotics and food supplements; (b) at least three members of a group consisting steviol glycoside, citric acid monohydrate, monosodium glutamate, glycine, mixtures and derivatives thereof; and (c) An apple-flavouring agent.
n. An apple flavoured horses' edible powder characterized by no residual chlorine taste comprising (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; (c) an apple-flavouring agent; and (d) prebiotic.

A non-limiting example for horse formulation is Px-E version 3, see Table 2)

Topper formulations: taste enhancers, formulations for enhancing palatably of commercially available dry food for pets.

These topper formulations comprise:
a. An isotonic taste enhancer, e.g., fluid, solid or gel-formed taste enhancer, edible meal topper, a treat tec., characterized by no residual chlorine taste for the hydration of mammals comprising at least three members of a group consisting steviol glycoside, citric acid monohydrate, monosodium glutamate, glycine, mixtures and derivatives thereof.
b. An isotonic taste enhancer characterized by no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.
c. An isotonic taste enhancer characterized by no residual chlorine taste for the hydration of mammals comprising at least three members of a group consisting of steviol glycoside, citric acid monohydrate, monosodium glutamate, glycine, mixtures and derivatives thereof.
d. An isotonic taste enhancer compositions characterized by no residual chlorine taste for the hydration of mammals comprising (a) at least three members of a group consisting steviol glycoside, citric acid monohydrate, monosodium glutamate, glycine, mixtures and derivatives thereof; and (b) prebiotic.
e. An isotonic taste enhancer characterized by chicken flavour and no residual chlorine taste for the hydration of mammals comprising at least three members of a group consisting steviol glycoside, citric acid monohydrate, monosodium glutamate, glycine, mixtures and derivatives thereof.
f. An isotonic taste enhancer characterized by chicken flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.
g. An isotonic taste enhancer characterized by fish flavour and no residual chlorine taste for the hydration of mammals comprising at least three members of a group consisting steviol glycoside, citric acid monohydrate, monosodium glutamate, glycine, mixtures and derivatives thereof.
h. An isotonic taste enhancer characterized by fish flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.
i. An isotonic taste enhancer characterized by beef flavour and no residual chlorine taste for the hydration of mammals comprising at least three members of a group consisting steviol glycoside, citric acid monohydrate, monosodium glutamate, glycine, mixtures and derivatives thereof.
j. An isotonic taste enhancer characterized by beef flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.

A non-limiting example for a topper formulation is XDG version 2 or version 3, see Table 3.

Ready-to-use liquid formulations—non-chlorine isotonic drink formulations.

These ready-to-use drink formulations comprise:
a. An isotonic drink characterized by no residual chlorine taste for the hydration of mammals comprising at least three members of a group consisting of steviol glycoside, citric acid monohydrate, monosodium glutamate, glycine, mixtures and derivatives thereof; and any derivatives and mixtures thereof.
b. An isotonic drink compositions characterized by no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.
c. An isotonic drink characterized by no residual chlorine taste for the hydration of mammals comprising (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof; and any derivatives and mixtures thereof.
d. An isotonic drink compositions characterized by no residual chlorine taste for the hydration of mammals comprising (a) a member of a group consisting of vaccines, iron-containing compositions, minerals, salts, amino acids, medicaments, vitamins, nutraceuticals, antibiotics and food supplements; and (b) at least three members of a group consisting steviol glycoside, citric acid monohydrate, monosodium glutamate, glycine, mixtures and derivatives thereof; and (c) prebiotic any derivatives and mixtures thereof.
e. An isotonic drink characterized by chicken flavour and no residual chlorine taste for the hydration of mammals comprising at least three members of a group consisting of steviol glycoside, citric acid monohydrate, monosodium glutamate, glycine, mixtures and derivatives thereof; and any derivatives and mixtures thereof.
f. An isotonic drink compositions characterized by chicken flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.
g. An isotonic drink characterized by fish flavour and no residual chlorine taste for the hydration of mammals comprising at least three members of a group consisting of steviol glycoside, citric acid monohydrate, monosodium glutamate, glycine, mixtures and derivatives thereof; and any derivatives and mixtures thereof.
h. An isotonic drink compositions characterized by fish flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.
i. An isotonic drink characterized by beef flavour and no residual chlorine taste for the hydration of mammals comprising at least three members of a group consisting of steviol glycoside, citric acid monohydrate, monosodium glutamate, glycine, mixtures and derivatives thereof; and any derivatives and mixtures thereof.
j. An isotonic drink compositions characterized by beef flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.

A non-limiting example for a ready-to-use liquid for dogs is X-D liver version 3, see Table 4. A non-limiting example for a ready-to-use liquid is an isotonic liquid for cats version 1, see Table 7.

Powder formulations: fast hydration palatable oral edible formulations.

These powder edible formulations comprise:
a. An edible powder characterized by no residual chlorine taste for the hydration of mammals comprising at least three members of a group consisting of steviol glycoside, citric acid monohydrate, monosodium glutamate, glycine, mixtures and derivatives thereof.
b. An edible powder characterized by no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.
c. An edible powder characterized by no residual chlorine taste for the hydration of mammals comprising at least three members of a group consisting of steviol glycoside, citric acid monohydrate, monosodium glutamate, glycine, mixtures and derivatives thereof.
d. An edible powder compositions characterized by no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.
e. An edible powder characterized by no residual chlorine taste for the hydration of mammals comprising (a) a member of a group consisting of vaccines, iron-containing compositions, minerals, salts, amino acids, medicaments, vitamins, nutraceuticals, antibiotics and food supplements; and (b) at least three members of a group consisting of steviol glycoside, citric acid monohydrate, monosodium glutamate, glycine, mixtures and derivatives thereof.
f. An edible powder characterized by no residual chlorine taste for the hydration of mammals comprising (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic any derivatives and mixtures thereof.
g. An edible powder characterized by no residual chlorine taste for the hydration of mammals comprising (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof.
h. An edible powder compositions characterized by no residual chlorine taste for the hydration of mammals comprising (a) a member of a group consisting of vaccines, iron-containing compositions, minerals, salts, amino acids, medicaments, vitamins, nutraceuticals, antibiotics and food supplements; (b) at least three members of a group consisting of steviol glycoside, citric acid monohydrate, monosodium glutamate, glycine, mixtures and derivatives thereof; and (c) prebiotic.
i. An edible powder characterized by chicken flavour and no residual chlorine taste for the hydration of mammals comprising at least three members of a group consisting of steviol glycoside, citric acid monohydrate, monosodium glutamate, glycine, mixtures and derivatives thereof.
j. An edible powder characterized by chicken flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.
k. An edible powder characterized by fish flavour and no residual chlorine taste for the hydration of mammals comprising at least three members of a group consisting of steviol glycoside, citric acid monohydrate, monosodium glutamate, glycine, mixtures and derivatives thereof.
l. An edible powder characterized by fish flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.
m. An edible powder characterized by beef flavour and no residual chlorine taste for the hydration of mammals comprising at least three members of a group consisting of steviol glycoside, citric acid monohydrate, monosodium glutamate, glycine, mixtures and derivatives thereof.
n. An edible powder characterized by beef flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.

A non-limiting example for a powder suitable for dogs is X54 (see Table 5). A non-limiting example for a powder suitable for cats is isotonic powder mix for cats version 1, see Table 8.

Gel formulations: edible isotonic gel formulations, including inter alia firm gel or shear-thinning thixotropic edible gel.

These edible gel formulations comprise:
a. an edible gel characterized by no residual chlorine taste for the hydration of mammals comprising at least three members of a group consisting of steviol glycoside, citric acid monohydrate, monosodium glutamate, glycine, mixtures and derivatives thereof.
b. an edible gel compositions characterized by no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.
c. an edible isotonic gel characterized by no residual chlorine taste for the hydration of mammals comprising at least three members of a group consisting of steviol glycoside, citric acid monohydrate, monosodium glutamate, glycine, mixtures and derivatives thereof.
d. an edible isotonic gel compositions characterized by no residual chlorine taste for the hydration of mammals comprising (a) at least three members of a group consisting steviol glycoside, citric acid monohydrate, monosodium glutamate, glycine, mixtures and derivatives thereof and (b) prebiotic any derivatives and mixtures thereof.
e. a solid shear thinning thixotropic edible gel characterized by no residual chlorine taste for the hydration of mammals comprising at least three members of a group consisting of steviol glycoside, citric acid monohydrate, monosodium glutamate, glycine, mixtures and derivatives thereof.
f. a solid shear thinning thixotropic edible gel characterized by no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.
g. an edible gel characterized by chicken flavour and no residual chlorine taste for the hydration of mammals comprising at least three members of a group consisting of steviol glycoside, citric acid monohydrate, monosodium glutamate, glycine, mixtures and derivatives thereof.
h. an edible gel compositions characterized by chicken flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.
i. an edible isotonic gel characterized by chicken flavour and no residual chlorine taste for the hydration of mammals comprising at least three members of a group consisting of steviol glycoside, citric acid monohydrate, monosodium glutamate, glycine, mixtures and derivatives thereof.
j. an edible isotonic gel compositions characterized by chicken flavour and no residual chlorine taste for the hydration of mammals comprising (a) at least three members of a group consisting steviol glycoside, citric acid monohydrate, monosodium glutamate, glycine, mixtures and derivatives thereof and (b) prebiotic any derivatives and mixtures thereof.
k. a solid shear thinning thixotropic edible gel characterized by chicken flavour and no residual chlorine taste for the hydration of mammals comprising at least three members of a group consisting of steviol glycoside, citric acid monohydrate, monosodium glutamate, glycine, mixtures and derivatives thereof.
l. a solid shear thinning thixotropic edible gel characterized by chicken flavour and no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.
m. an edible gel characterized by fish flavour and no residual chlorine taste for the hydration of mammals comprising at least three members of a group consisting of steviol glycoside, citric acid monohydrate, monosodium glutamate, glycine, mixtures and derivatives thereof.
n. an edible gel compositions characterized by fish flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.
o. an edible isotonic gel characterized by fish flavour and no residual chlorine taste for the hydration of mammals comprising at least three members of a group consisting of steviol glycoside, citric acid monohydrate, monosodium glutamate, glycine, mixtures and derivatives thereof.
p. an edible isotonic gel compositions characterized by fish flavour and no residual chlorine taste for the hydration of mammals comprising (a) at least three members of a group consisting steviol glycoside, citric acid monohydrate, monosodium glutamate, glycine, mixtures and derivatives thereof and (b) prebiotic any derivatives and mixtures thereof.
q. a solid shear thinning thixotropic edible gel characterized by fish flavour and no residual chlorine taste for the hydration of mammals comprising at least three members of a group consisting of steviol glycoside, citric acid monohydrate, monosodium glutamate, glycine, mixtures and derivatives thereof.
r. a solid shear thinning thixotropic edible gel characterized by fish flavour and no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.
s. an edible gel characterized by beef flavour and no residual chlorine taste for the hydration of mammals comprising at least three members of a group consisting of steviol glycoside, citric acid monohydrate, monosodium glutamate, glycine, mixtures and derivatives thereof.
t. an edible gel compositions characterized by beef flavour and no residual chlorine taste for the hydration of mammals comprising at least three members of a group consisting (a) steviol glycoside, citric acid monohydrate, monosodium glutamate, glycine, mixtures and derivatives thereof; and (b) prebiotic any derivatives and mixtures thereof.
u. an edible isotonic gel characterized by beef flavour and no residual chlorine taste for the hydration of mammals comprising at least three members of a group consisting of steviol glycoside, citric acid monohydrate, monosodium glutamate, glycine, mixtures and derivatives thereof.
v. an edible isotonic gel compositions characterized by beef flavour and no residual chlorine taste for the hydration of mammals comprising (a) at least three members of a group consisting steviol glycoside, citric acid monohydrate, monosodium glutamate, glycine, mixtures and derivatives thereof and (b) prebiotic any derivatives and mixtures thereof.
w. a solid shear thinning thixotropic edible gel characterized by beef flavour and no residual chlorine taste for the hydration of mammals comprising at least three members of a group consisting of steviol glycoside, citric acid monohydrate, monosodium glutamate, glycine, mixtures and derivatives thereof.
x. a solid shear thinning thixotropic edible gel characterized by beef flavour and no residual chlorine taste for the hydration of mammals comprising (a) at least three members of a group consisting steviol glycoside, citric acid monohydrate, monosodium glutamate, glycine, mixtures and derivatives thereof; and (b) prebiotic any derivatives and mixtures thereof.

A non-limiting example for an edible gel is firm-gel carrageenan—LBG, see Table 6.

### Example 1

Tonisity Equine-Px-E version 3, see Table 2.

Problem: Horses that are subjected to long bouts of exercise will lose massive amounts of water and electrolytes. If the horse is not quickly rehydrated, this can become a serious emergency and lead to kidney failure.

Delivery: Powder

Properties: White color (closely resembles Tonisity Px); Pleasant apple aroma and flavouring; and Oral rehydration formula.

Uses: Dry top dressing sprinkled on feed; Mixed with water to create an isotonic solution; Mixed with other feeds (e.g., bran, beet pulp, etc.) to make a mash

Results: In trials, the mash and top dressing were very well received

Trials indicated that horses had better exercise tolerance, improved stamina and demeanor, and generally more energy.

Intended Uses: For working horses performing heavy exercise; after 3-day events; dressage horses; and Long distance riding and racing.

Package Size Suggestions: 155 g scoop of the powder per horse per day, so package could be 1 or 2.5 kg.

TABLE 2
The ingredients of a preferred embodiment for
a horse nourishing formulation, Px-E version 3
Px-E Version 3

| | Solution | Dry Mix |
|---|---|---|
| Ingredients | % | % |
| Demineralised Water | 97.000 | |
| Glucose Anhydrous | 1.674 | 55.800 |
| Sodium Chloride | 0.220 | 7.334 |
| Potassium Chloride | 0.150 | 5.000 |
| Glycine | 0.150 | 5.000 |
| Xanthan Gum | 0.050 | 1.667 |
| Monosodium Phosphate | 0.100 | 3.333 |
| Citric Acid Anhydrous | 0.060 | 2.000 |
| Hydrolysed Whey Protein Isolate | 0.150 | 5.000 |
| L-Glutamic Acid | 0.040 | 1.333 |
| Monosodium Glutamate | 0.390 | 13.000 |
| Rebaudioside A 98% Min. | 0.010 | 0.333 |
| Thaumatin | 0.003 | 0.100 |
| Apple Flavour Maxarome Ac 1632 | 0.003 | 0.100 |
| TOTAL | 100.000 | 100.000 |

### Example 2

Topper (Gravy) for dogs-Gravy topper, XDG version 2 and version 3, see Table 3. The difference between two formulations is a higher viscosity in version 3 (XDG3).

Problem: Dry food historically has lacked palatability for dogs. To maintain a healthy physique and internal equilibrium, a tasty and vitamin-rich “meal topper” can be poured over the dry food. These gravies can come with different flavouring that targets a specific health improvement such as joint and bone, shiny coat, etc.

Delivery: Gravy/Puree

Properties: Prebiotic formula (oligosaccharides); Rich, meaty flavour with a thicker consistency; Target Audience: Offers a more diversified diet and a meal of pleasure for all dogs. Recreational usage and also to enhance the quality of the less healthy food; Older dogs who have trouble drinking enough water

Benefits and Uses: Palatable flavour for dogs, delivering nutrients and vitamins through an isotonic delivery system; and Ready to use—no preparation required before serving, just pour on top of standard dry food.

TABLE 3
The ingredients of a preferred embodiment for
a topper formulation, XDG version 2 and version 3.
Gravy Topper XDG

| | XDG V2 | XDG V3 |
|---|---|---|
| Ingredients | % | % |
| Demineralized Water | 95.04 | 94.84 |
| Glucose Anhydrous | 1.60 | 1.60 |
| Sodium Chloride | 0.22 | 0.22 |
| Potassium Chloride | 0.15 | 0.15 |
| Glycine | 0.15 | 0.15 |
| Xanthan Gum | 0.20 | 0.40 |
| Monosodium Phosphate | 0.10 | 0.10 |
| Liquid Liver Hydrolisate | 2.00 | 2.00 |
| Oligosaccharide | 0.10 | 0.10 |
| L-Glutamic Acid | 0.04 | 0.04 |
| Monosodium Glutamate | 0.19 | 0.19 |
| Burnt Sugar | 0.20 | 0.20 |
| Rebaudioside (A Steviol Glycoside A | 0.01 | 0.01 |
| 98% Min. | | |
| Total | 100.00 | 100.00 |

Preparation: premix all dries before addition to water; pH 3.8-4.2 at 20 degrees C.

### Example 3

Ready-to-use liquid for dogs: X-D Liver hydrosylate version 3 (see Table 4).

Problem: Most dogs do not prefer to drink tap water that contains chlorine. Both chlorine and ammonia are products that not only give off strong odors that dogs hate, but are also highly harmful to them; see https://www.animalwised.comAop-10-smells-dogs-hate-1568.html; incorporated herein as reference. Additionally, there does not exist any hydration liquid in the pet retail market aimed at providing nutrients and electrolyte replenishment to cats. The industry is hyper-focused on food products, leaving a huge missed opportunity for liquids. This RTU liquid can only be compared to Oralade (sold only in Europe), from which this formula is derived.

Delivery: Liquid

Properties: Fast hydration with an isotonic delivery system; Ready-to-use and palatable oral hydration liquid; Pleasant chicken aroma and taste; and Prebiotic formula (oligosaccharides)

Audience: All dogs, especially those who have been exercising, working or are recovering from illness. Benefits and Uses: Ready to use formula with no mixing required; Provides fast and efficient hydration while promoting gut health through simple amino acids and prebiotics with an isotonic delivery system; Recommended for pets requiring rehydration due to recreational activities: exercise, travel, hot weather; and Convenient when on-the-go; Packaging Sizes: Could have a variety of these.

TABLE 4
The ingredients of a preferred embodiment
for a topper formulation, XDG version 2 and version 3.
Px D Liquid Liver Hydrolisate Version 3

| | XDV3 |
|---|---|
| Ingredients | % |
| Demineralized Water | 95.32 |
| Glucose Anhydrous | 1.60 |
| Sodium Chloride | 0.22 |
| Potassium Chloride | 0.15 |
| Glycine | 0.15 |
| Monosodium Phosphate | 0.10 |
| Xanthan Gum | 0.12 |
| Mercey C14028 Liver Hydrolisate | 2.00 |
| Oligosaccharide | 0.10 |
| L-Glutamic Acid | 0.04 |
| Monosodium Glutamate | 0.19 |
| Rebaudioside A 98% Min | 0.01 |
| Total | 100.00 |

### Example 4

Powder for dogs: X54 spray dried liver hydrolisate version 2, see Table 5.

Problem: Inconvenient and heavy water bottles make it more difficult to take your dog to the outdoors and for recreational exercise. These lightweight single servings make it easy to deliver electrolytes and rehydration to your dog.

Delivery: Powder

Properties: Fast hydration with an isotonic delivery system; Ready-to-use and palatable oral hydration powder; Pleasant chicken aroma and taste; and Prebiotic formula (oligosaccharides)

Benefits and Uses: Mixes readily with water, light shaking required; Very portable and lightweight: great for traveling, hiking, camping, exercise; Provides fast and efficient hydration while promoting gut health through simple amino acids and prebiotics with an isotonic delivery system; Recommended for pets requiring rehydration due to recreational activities: exercise, travel, hot weather; and Extremely long shelf life (longer than the Gel and RTU Liquid)

Packaging Sizes: Single serving (mix with 500 mL of water), similar to Crystal Light.

How to Use: Mix the powder with 500 mL of water and pour into an empty bowl.

TABLE 5
The ingredients of a preferred embodiment for a powder for dogs
formulation, X54 Spray Dried Liver Hydrolisate Version 2.

| | Liquid | Powder |
|---|---|---|
| Ingredients | % | % |
| Demineralized water | 96.50 | |
| Glucose anhydrous | 1.82 | 52.000 |
| Sodium chloride | 0.22 | 6.286 |
| Potassium chloride | 0.15 | 4.286 |
| Glycine | 0.15 | 4.286 |
| Monosodium phosphate | 0.10 | 2.856 |
| Citric acid anhydrous | 0.22 | 6.286 |
| Spray dried liver hydrolisate | 0.50 | 14.286 |
| Oligosaccharide | 0.10 | 2.857 |
| L-glutamic acid | 0.04 | 1.143 |
| Monosodium glutamate | 0.19 | 5.428 |
| Rebaudioside a 98% min. | 0.01 | 0.286 |
| Total | 100.000 | 100.000 |

### Example 5

Gel for dogs: firm gel carrageenan-LBG, (LBG—locust bean gum), see Table 6.

Problem: Providing water to your dog on-the-go requires a portable water bowl, which is inconvenient to carry and use. This palatable hydration gel can be administered on surfaces like grass and sidewalk and require no additional equipment.

Delivery: Gel

Properties: Using an isotonic delivery system (which occurs when the gel reaches the dog's stomach), the gel provides electrolytes and hydration to your dog when they need it most; Provides fast and efficient hydration while promoting gut health through simple amino acids and prebiotics (oligosaccharides) with an isotonic delivery system; and pleasant chicken liver aroma and taste.

Benefits: When walking your dog, no water bowl is required. The gel can be administered on any surface (grass, sidewalk, etc.); Great for recreational exercising, especially in hot weather climates; Stable in a variety of temperature conditions; and Easy to carry and convenient.

Packaging Sizes: Foil bag (250 mL), single serving, or peel-wrapped cubes.

How to Use: Squeeze foil bag onto any surface (grass, sidewalk, etc.). The number of cubes given would depend on the size of the dog.

TABLE 6
The ingredients of a preferred embodiment for an edible
gel for dogs formulation, Prototype Firm Gel Carrageenan-LBG.
Prototype Firm Gel Carrageenan-LBG

| Ingredients | % |
|---|---|
| Demineralized Water | 95.450 |
| Glucose Anhydrous | 1.690 |
| Sodium Chloride | 0.220 |
| Potassium Chloride | 0.150 |
| Glycine | 0.150 |
| Kappa Carrageenan Gum | 0.600 |
| Locust Bean Gum | 0.600 |
| Xanthan Gum | 0.200 |
| Oligosaccharide | 0.100 |
| Spray Dried Chicken Liver | 0.500 |
| L-Glutamic Acid | 0.040 |
| Monosodium Glutamate | 0.190 |
| Burnt Sugar Syrup | 0.100 |
| Rebaudioside A Min 98% | 0.010 |
| Total | 100.000 |

Preparation for gel: Premix all dries before addition to water

Disperse and dissolve in water at max 35 degrees C.

Fill into pouches, double seal, immerse for 30 minutes minimum in boiling water. Remove from boiling water, allow to cool.

Additional non-limiting embodiment of gel consists agar and pectin as alternatives to Carrageenan—LBG gelling composition.

### Example 6

Ready-to-use liquid for cats: Isotonic liquid for cats, see Table 7.

Problem: Cats are notoriously picky about drinking water. Electrophysiological recordings show that water is not tasteless to cats; see Bartoshuk, L. M., M. A. Harned, and L. H. Parks. “Taste of water in the cat: effects on sucrose preference.” Science 171.3972 (1971): 699-701.disclosed herein as a reference. However, there does not exist any hydration liquid in the pet retail market aimed at providing nutrients and electrolyte replenishment to cats. The industry is hyper-focused on food products, leaving a huge missed opportunity for liquids. This RTU liquid can only be compared to Oralade (sold only in Europe), from which this formula is derived.

Delivery: Liquid

Properties: Fast hydration with an isotonic delivery system; Ready-to-use and palatable oral hydration liquid; Pleasant chicken or fish aroma and taste; and Prebiotic formula (oligosaccharides).

Benefits and Uses: Ready to use formula with no mixing required; Provides fast and efficient hydration while promoting gut health through simple amino acids and prebiotics with an isotonic delivery system; recommended for pets requiring rehydration due to recreational activities: exercise, travel, hot weather; and Convenient when on-the-go.

Packaging Sizes: Could have a variety of sizes.

How to Use: Pour the contents into an empty bowl.

TABLE 7
The ingredients of a preferred embodiment for an isotonic
drink for cats' formulation, Isotonic Liquid for Cats Version 1.
Isotonic Liquid For Cats Version 1

| Ingredients | % |
|---|---|
| Demineralized Water | 94.20 |
| Glucose Anhydrous | 1.50 |
| Sodium Chloride | 0.15 |
| Potassium Chloride | 0.10 |
| Glycine | 0.30 |
| Monosodium Phosphate | 0.10 |
| Xanthan Gum | 0.12 |
| Liquid Liver Hydrolysate | 3.00 |
| Fructo-Oligosaccharide | 0.10 |
| L-Glutamic Acid | 0.04 |
| Monosodium Glutamate | 0.19 |
| Taurine | 0.08 |
| Liquid Roast Chicken Flavour | 0.12 |
| Total | 100.00 |

### Target pH 3.3-3.6 At 20 Degrees C.

### Example 7

Powder for cats: Isotonic edible powder mix for cats, see Table 8.

Problem: It is important to keep your cat hydrated, particularly in high temperature conditions. Particularly if they spend time in the outdoors and in direct sunlight, combined with their meager daily intake of water, an electrolyte replenishment formula can be a critical tool to increase hydration. This is also a good product for older cats who have trouble drinking water or critical care patients who just underwent some medical procedure.

Delivery: Powder

Properties: Fast hydration with an isotonic delivery system; Ready-to-use and palatable oral hydration powder; Pleasant chicken of beef aroma and taste; and Prebiotic formula (oligosaccharides).

Audience: Older cats that have trouble drinking, post-trauma, but also for recreational usage.

Benefits and Uses: Mixes readily with water, light shaking required; Very portable and lightweight; Provides fast and efficient hydration while promoting gut health through simple amino acids and prebiotics with an isotonic delivery system; and Extremely long shelf life (longer than the Gel and RTU Liquid)

Packaging Sizes: Single serving (mix with 100 mL of water)

How to Use: Mix the powder with 100 mL of water and pour into an empty bowl.

TABLE 8
The ingredients of a preferred embodiment for an isotonic
powder for cats formulation, Isotonic Powder Mix For Cats Version 1.

| Isotonic Powder Mix for Cats Version 1 | % | % |
|---|---|---|
| Ingredients | Liquid | Powder |
| Demineralized Water | 96.00 | |
| Glucose Anhydrous | 1.34 | 33.50 |
| Sodium Chloride | 0.20 | 5.00 |
| Potassium Chloride | 0.15 | 3.75 |
| Glycine | 0.30 | 7.50 |
| Monosodium Phosphate | 0.10 | 2.50 |
| Citric Acid Anhydrous | 0.30 | 7.50 |
| Spray Dried Liver Hydrolysate | 1.00 | 25.00 |
| Fructo-Oligosaccharide | 0.10 | 2.50 |
| L-Glutamic Acid | 0.04 | 1.00 |
| Monosodium Glutamate | 0.19 | 4.75 |
| Taurine | 0.08 | 2.00 |
| Roast Chicken Flavour Powder | 0.20 | 5.00 |
| Total | 100.00 | 100.00 |

1.-81. (canceled)

82. An oral isotonic composition for the hydration of mammals, said composition comprises:
(a) at least one sweetener;
(b) at least one electrolyte; and
(c) at least one amino acid, wherein said amino acids is glutamate.

83. A hydration composition comprise water and a hydration additive, said hydration additive comprises:
at least one sweetener,
at least one electrolyte; and
at least one amino acid,
wherein one said amino acid is glutamate, further wherein said hydration additive is present in the hydration composition in an amount of about 0.1% to about 10% by weight.

84. The oral isotonic composition of claim 82, said composition comprises (a) at least three members of a group consisting steviol glycoside, citric acid monohydrate, monosodium glutamate, glycine, mixtures and derivatives thereof; and (b) prebiotic any derivatives and mixtures thereof.

85. The oral isotonic composition of claim 84, wherein said prebiotics comprises oligosaccharides or fructo-oligosaccharides.

86. The oral isotonic composition of claim 82, wherein said composition is an isotonic drink

87. The oral isotonic composition of claim 82, wherein said sweeteners are selected from a group consisting of aspartame, dextrose, glucose, saccharin, steviol glycosides, and thaumatin,

88. The oral isotonic composition of claim 82, wherein said electrolytes are selected from a group consisting of potassium, sodium, chloride, phosphate and any combination thereof.

89. The oral isotonic composition of claim 82, wherein said amino acids are selected from a group consisting of taurine, glutamic acid, glycine and nay combination thereof.

90. The oral isotonic composition of claim 82, said composition further comprises maltodextrins, dextrose and starches as sources of readily digestible carbohydrates.

91. The oral isotonic composition of claim 82, said composition is selected from a group consisting of edible gel, edible powder, edible isotonic gel, a solid shear thinning thixotropic edible gel and any combination thereof.

92. The oral isotonic composition of claim 82, wherein said composition is further characterized by one of flavours selected from palatants consisting of meat-flavoured protein hydrolysates, chicken flavour, fish flavour, beef flavour, apple-flavour, fruit flavours, vegetable flavours, and any combination thereof.

93. The oral isotonic composition of claim 82, wherein said composition further comprises (a) a member of a group consisting of vaccines, iron-containing compositions, minerals, salts, amino acids and arginine, medicaments, vitamins, nutraceuticals, antibiotics and food supplements; and (b) at least three members of a group consisting of steviol glycoside, citric acid monohydrate, monosodium glutamate, glycine, mixtures and derivatives thereof.

94. The oral isotonic composition of claim 82, wherein said composition is selected from a group consisting of edible gel, edible powder, edible isotonic gel, a solid shear thinning thixotropic edible gel and any combination thereof.

95. A method for improving water intake and hydration in an animal comprising:
administering a hydration composition to the animal,
wherein the hydration composition comprises water and a hydration additive, further wherein the hydration additive consists essentially of a combination of sweeteners, electrolytes and glutamate.
